(19) United States
(12) Patent Application Publication
(10) Pub. No.: US 2021/0361923 A1
(43) Pub. Date: Nov. 25, 2021

(54) PORTABLE RETRACTING DEVICE FOR MEDICAL TUBING MANAGEMENT

(71) Applicant: Dax Hamilton, Aumsville, OR (US)

(72) Inventor: Dax Hamilton, Aumsville, OR (US)

(21) Appl. No.: 16/879,570

(22) Filed: May 20, 2020

(51) Int. Cl. A61M 39/08; B65H 75/44

(52) U.S. Cl. CPC A61M 39/08; A61M 2039/0009; B65H 75/4471

(57) ABSTRACT

A portable retracting device for medical tubing management includes a housing. The housing includes a retractable cord disposed within the housing, wherein the retractable cord is configured to extend from a side of the housing. The device includes a first spring clamp configured to attach to clothing of an individual, a second spring clamp configured to clamp to the medical tube having a first diameter at a first location of the first medical tube, and a third spring clamp coupled to an end of the retractable cord configured to clamp the medical tube at a second location of the first medical tube to form a first loop in the first medical tube from the first location of the first medical tube to the second location of the first medical tube.

### BACKGROUND

It is common for a patient, in a hospital environment, to have one or more medical tubes (e.g., intravenous (IV) tube) connected to the patient for medical treatment. For example, a medical tube may be connected to the patient at a first end and to a medical device (e.g., an IV bag) at a distal end. Medical tubes may become tangled with one another which may negatively affect the treatment of the patient.

Additionally, a medical tube typically includes slack to allow for the patient to freely move with respect to the medical tube connected to the patient at the first end and the medical device connected to the patient at the second end. The slack enables at least a portion of the medical tube to lie on a floor. As such, a patient may trip over the medical tube. A rolling medical device (e.g., IV pole) may roll over the medical tube and become entangled with the medical tube. The entangling of a patient and/or medical device with a medical tube may cause undue stress on the medical tube, may pull the medical tube away from a patient and/or medical device, and the like. This may increase the risk of infection and/or create a life-threatening medical situation.

### BRIEF DESCRIPTION OF DRAWINGS

The present description will be understood more fully from the detailed description given below and from the accompanying drawings of various embodiments of the present embodiment, which description is not to be taken to limit the present embodiment to the specific embodiments but are for explanation and understanding. Throughout the description the drawings may be referred to as drawings, figures, and/or FIGS.

FIG. 1A illustrates a medical tube management system with a portable retracting device in a retracted state, according to an embodiment.

FIG. 1B illustrates a top view a portable retracting device in a retracted state, according to an embodiment.

FIG. 1C illustrates a perspective view of a portable retracting device in a retracted state illustrated in FIG. 1B, according to an embodiment.

FIG. 1D illustrates a bottom view of a portable retracting device in an extended state illustrated in FIG. 1B, according to an embodiment.

FIG. 2 illustrates a top view of a portable retracting device including a reel and a retractable cord, according to an embodiment.

FIG. 3A illustrates a side view of a tube clamp of a portable retracting device, according to an embodiment.

FIG. 3B illustrates a perspective view of a tube clamp of a portable retracting device, according to an embodiment.

FIG. 3C illustrates a side view of a tube clamp of a portable retracting device clamping a medical tube, according to an embodiment.

FIG. 3D illustrates a side view of a tube clamp of a portable retracting device clamping a medical tube, according to an embodiment.

FIG. 4 illustrates a medical tube disposed on a ground surface, according to an embodiment.

FIG. 5A illustrates a medical tube management system with a portable retracting device in a retracted state, according to an embodiment.

FIG. 5B illustrates a medical tube management system with a portable retracting device in an extended state, according to an embodiment.

FIG. 6 illustrates a method of medical tube management, according to an embodiment.

### DETAILED DESCRIPTION

Methods, devices and systems related to managing medical tubing as disclosed herein will become better understood through a review of the following detailed description in conjunction with the figures. The detailed description and figures provide merely examples of the various embodiments described herein. Those skilled in the art will understand that the disclosed examples may be varied, modified, and altered and not depart from the scope of the embodiments described herein. Many variations are contemplated for different applications and design considerations; however, for the sake of brevity, the contemplated variations may not be individually described in the following detailed description.

Throughout the following detailed description, example embodiments of various methods, devices and systems for managing medical tubing are provided. Related elements in the example embodiments may be identical, similar, or dissimilar in different examples. For the sake of brevity, related elements may not be redundantly explained in multiple examples except to highlight dissimilar features. Instead, the use of a same, similar, and/or related element names and/or reference characters may cue the reader that an element with a given name and/or associated reference character may be similar to another related element with the same, similar, and/or related element name and/or reference character in an example embodiment explained elsewhere herein. Elements specific to a given example may be described regarding that particular example embodiment. A person having ordinary skill in the art will understand that a given element need not be the same and/or similar to the specific portrayal of a related element in any given figure or example embodiment in order to share features of the related element.

As used herein “same” means sharing all features and “similar” means sharing a substantial number of features or sharing materially important features even if a substantial number of features are not shared. As used herein “may” should be interpreted in the permissive sense and should not be interpreted in the indefinite sense. Additionally, use of “is” regarding embodiments, elements, and/or features should be interpreted to be definite only regarding a specific embodiment and should not be interpreted as definite regarding the invention as a whole. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, and Abstract.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not be redundantly labeled. A person having ordinary skill in the art will recognize based on the disclosure herein redundant and/or duplicated elements of the same FIG. Despite this, redundant labeling may be included where helpful in clarifying the structure of the depicted example embodiments.

A patient may be connected to one or more medical tubes (e.g., oxygen tube, IV tubes, etc.). The medical tubes are connected to the patient (e.g., via an IV port, an oxygen mask, etc.) at one end of the tube and to a medical device (e.g., an IV pump, oxygen pump, IV bag, etc.) at an opposite second end of the medical tube. One or more medical tubes may become entangled with one another, or may become entangled with the patient, a medical professional and/or a medical device.

A medical tube may become pinched when it is entangled. This may limit or prohibit fluid (e.g., medicine, oxygen, etc.) from passing through the medical tube and subsequently administered to the patient. Entanglement of a medical tube may cause the medical tube to become detached from the patient, for example, at an IV port. This may increase the risk of infection at the site of the IV port. Additionally, medicine flowing through the medical tube would be prohibited from being administered to the patient at the IV port which may cause a life-threatening medical issue.

Similarly, entanglement of a medical tube may cause the medical tube to become detached from a medical device (e.g., medical pump, oxygen pump, IV pump, IV bag, IV device, etc.). Accordingly, medicine would be unable to be pumped through the medical tube and subsequently be administered to the patient which may cause a life-threatening medical issue.

Implementations of the disclosure address the above-mentioned deficiencies and other deficiencies by providing methods, systems, devices, or apparatuses to manage medical tubing. In one embodiment, a retractable medical device is configured to clamp onto a medical tube and create a loop in the medical tube that is suspended above a ground surface. Accordingly, the medical tube does not contact the ground surface which reduces the risk entanglement.

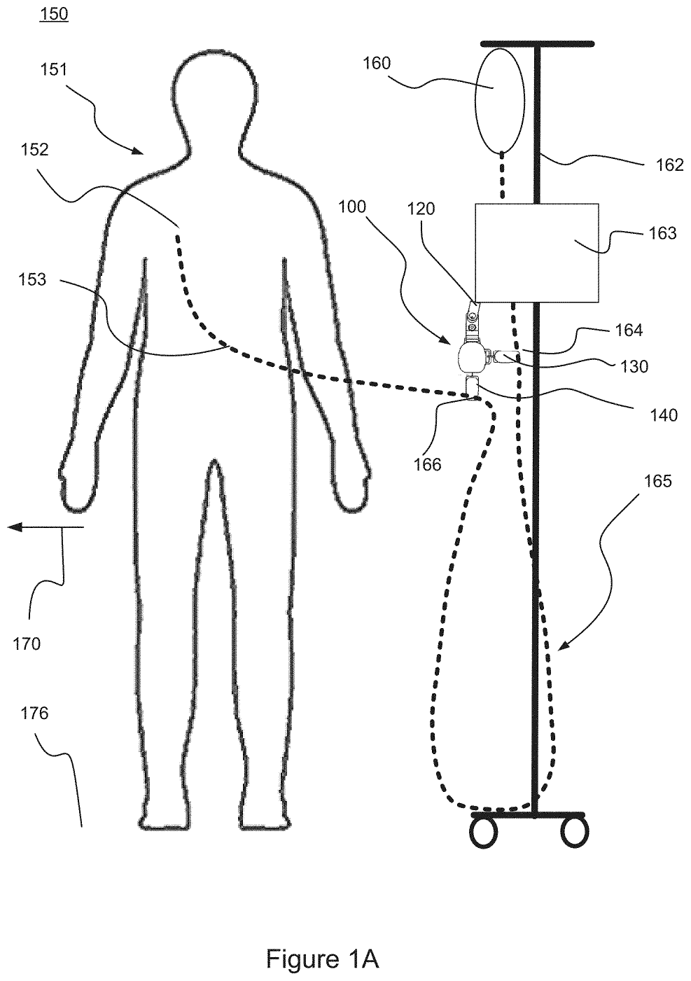

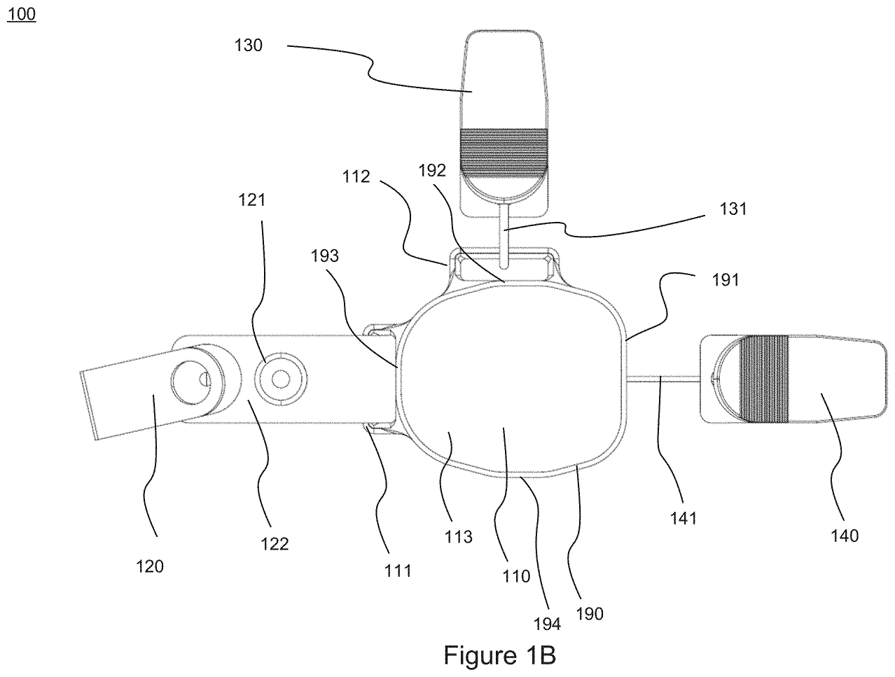

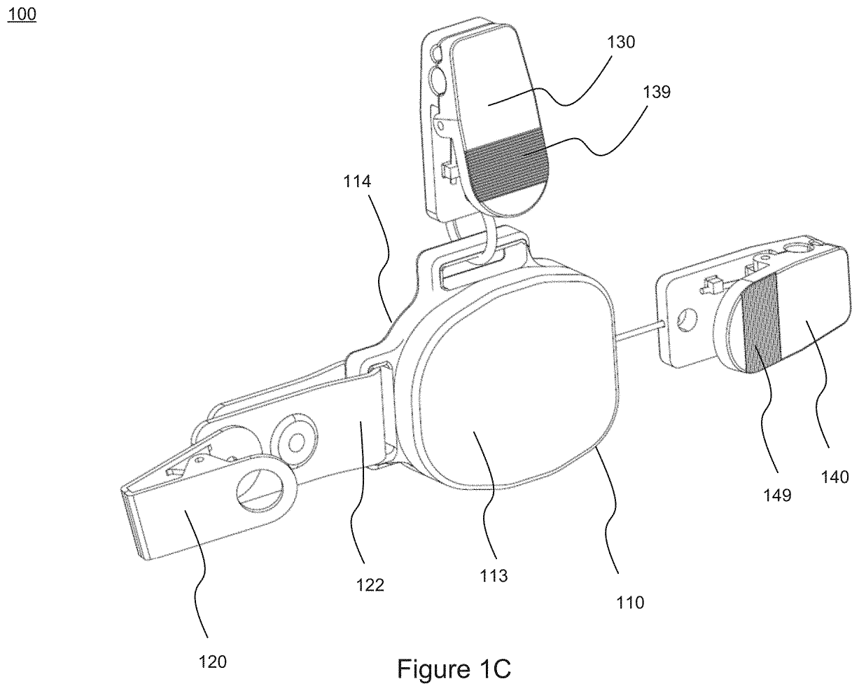

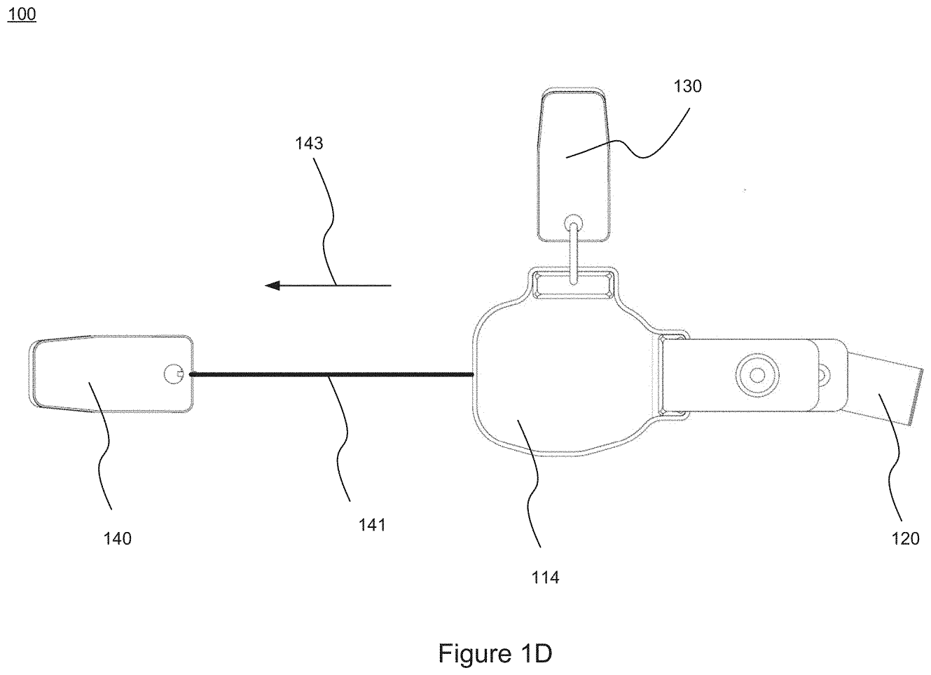

A FIG. 1A depicts an embodiment of a medical tubing management system 150 (also referred to as a “system 150”). In particular, the FIG. 1A depicts a device 100 of the system 150 in a retracted state.

The system 150 includes a medical tube 153 and a device 100. The medical tube 153, in one embodiment, is an IV tube coupled to an individual 151 at a location 152 (e.g., an IV port) of the individual (at a first distal end of the tube 153) and to a medical device at an opposite second distal end of the tube. For example, the tube 153 is coupled to the infusion pump 163 and/or the IV bag 160. The infusion pump 163 and the IV bag 160 may be coupled to the IV pole 162 that is able to roll along the ground surface 176. Moreover, the IV bag and/or the infusion pump are fluidically coupled to the individual.

The device 100 is coupled to the individual 151 via the clamp 120. For example, the clamp 120 may be coupled to clothing or a belt of the individual 151. The device 100 may be coupled at a vertical position between the location 152 and the ground surface 176.

The clamp 130 is clamped to the tube 153 (at the location 164 of the tube 153). The clamp 130 can be clamped at any position of the tube 153 such that there is slack in the tube 153 between the location 152 of the individual and the location 164. The slack limits the amount of stress acting on the tube at the location 152 of the individual in response to a pulling force acting on the tube.

The clamp 140 may be clamped to the tube 153 (at the location 166 of the tube 153). The loop 165 of the tube 153 may be formed between the location 164 and the location 166 of the tube 153. The loop 165 may be suspended above the ground surface 176. Accordingly, the loop 165 cannot be entangled at or near the ground surface 176. For example, wheels of the IV pole 162 and/or feet of the individual 151 cannot become entangled with the tube 153 near the ground surface 176. The clamp 140 can be clamped to the tube 153 at any location such that a loop is created (between the clamp 130 and the clamp 140) and suspended above the ground surface 176. Additionally, it should be appreciated that at least a portion of the tube 153 may be on the ground surface 176 prior to formation of the loop 165. For example, prior to formation of the loop 165, a portion of the tube 153 lays along the ground surface 176 and causes a tripping or entanglement hazard at the ground surface 176.

A pulling force may be acted on the tube 153, such as in the direction 170. For example, the individual 151 walks away from the IV pole 162 causing a pulling force to act on the tube 153 in the direction 170. Accordingly, the clamp 120 remains coupled to the individual 151 and the clamp 130 remains coupled to the tube at the location 164.

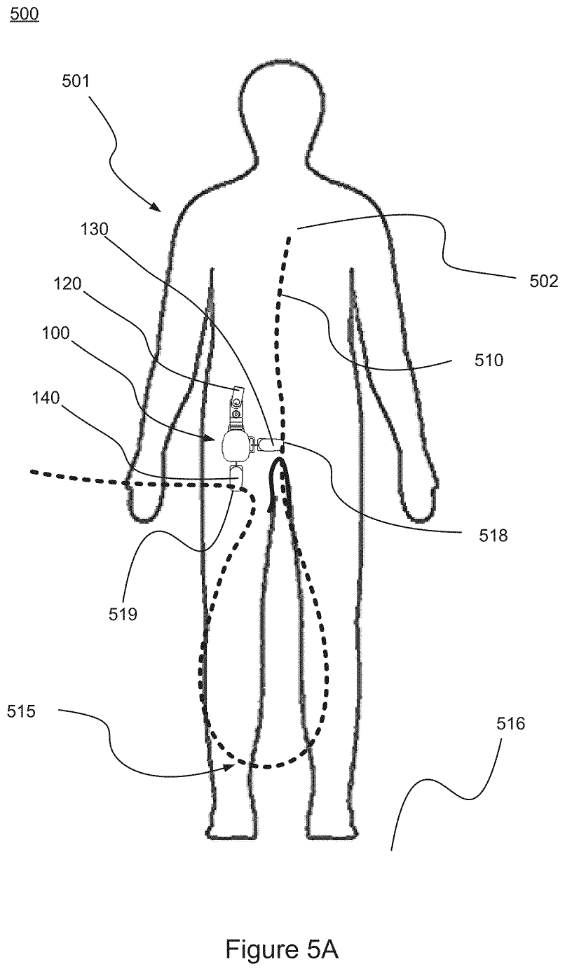

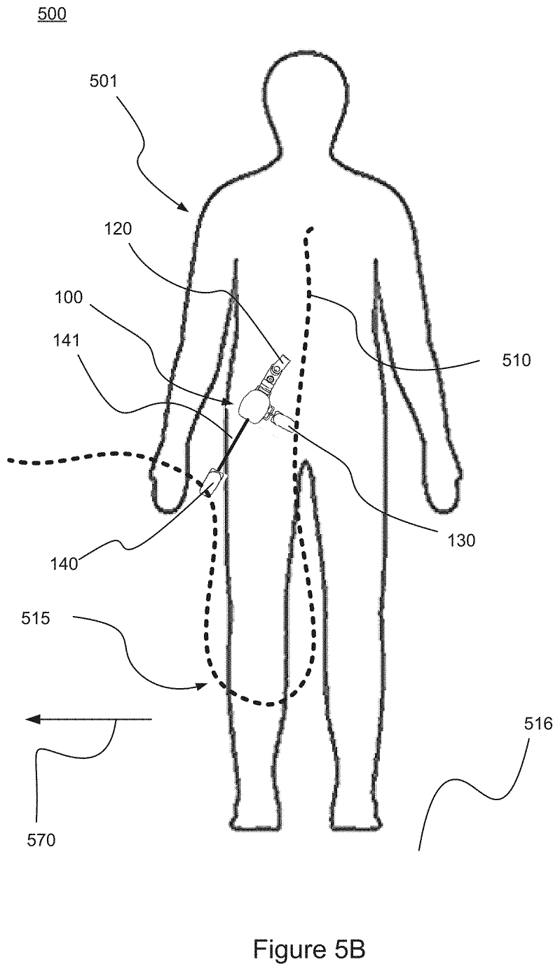

Additionally, the clamp 140 extends away from the housing 110 of the device 100 via the cord 141 (see FIG. 5B described in further detail herein). Accordingly, the pulling force is absorbed via the clamp 140 extending from the housing 110 (and the clamp 120 clamped to the individual and the clamp 130 clamped to the tube 153) and the pulling force does act on the tube 153 at the location 152 of the individual 151. For example, this prohibits the tube from pulling out of the individual at an IV port at the location 152. Moreover, the loop 165 remains suspended above the ground surface 516.

In response to the pulling force not acting on the tube 153 in the direction 170 (for example, the individual walks back towards the IV pole 162), the clamp 140 is drawn towards the housing 110 of the device 100 via the cord 141 retracting back into the housing of device 100. Moreover, the loop 165 remains suspended above the ground surface 176.

FIG. 1B depicts the portable retracting device 100 (also referred herein as the “device 100”) for medical tubing management, according to an embodiment. In particular, the FIG. 1B depicts a top view of the device 100. The device 100 may be configured to facilitate in the management of medical tubing such that the medical tubing does not become entangled.

The device 100 includes the housing 110, the first clamp 120 (also referred to herein as the “clamp 120”) configured to clamp to a patient or object proximate the patient, the second clamp 130 (also referred to herein as the “clamp 130”) configured to clamp on to a medical tube at a first location, and the third clamp 140 (also referred to herein as the “clamp 140” or a “coupling device”) configured to clamp on to a medical tube at a second location.

The housing 110 of the device 100 includes a periphery 190. The periphery 190 includes a first side 191, a second side 192, a third side 193 and a fourth side 194. The sides of the device 100 to form periphery 190 may also be referred to as walls. Moreover, in various embodiments, each side is approximately 90° with respect to an adjacent side. For example, first side 191 is positioned approximately 90° from the second side 192 and positioned approximately 90° from the fourth side 194.

The housing 110 of the device 100 includes the retractable cord 141 coupled to the clamp 140. The retractable cord 141 extends from side 191. The FIG. 1B depicts the retractable cord 141 in a retracted state. In one embodiment, the retractable cord 141 may be retracted within the housing 110 via a reel, which will be described in further detail herein. Referring to the FIG. 1D, the retractable cord 141 is in an extended state. For example, in response to a force acting on the retractable cord 141, in the direction 143, the retractable cord 141 is extended from the housing 110. Upon a force not acting on the retractable cord 141 (while in the extended state), the retractable cord 141 is automatically retracted into the housing 110 via a reel.

Referring again to the FIG. 1B, the housing 110 includes the loop 111 for coupling the clamp 120 to the housing 110. In one embodiment, the clamp 120 is removably coupled to the leash 122. The clamp 120 extends from side 193. Leash 122 is removably coupled to the loop 111. For example, the leash 122 may extend through loop 111 and may be retained on loop via snap 121. The leash 122 may pivot with respect to the loop 111. In various embodiments, the leash 122 may include various materials, such as but not limited to, plastic, leather and the like.

The leash 122 allows the clamp 120 to be coupled to the housing 110 and allows the clamp 120 to pivot with respect to the housing 110. As such, the clamp 120 may pivot with respect the loop 111. Additionally, the clamp 120 is pivotably coupled to the leash 122. As such, the clamp 120 may rotate with respect to the leash 122.

The housing 110 includes the ring 131 for coupling the clamp 130 to housing 110. In one embodiment, the clamp 130 extends from the side 192. In one embodiment, the clamp 130 is coupled to the ring 131 and the ring 131 is coupled to the loop 112. The ring 131 allows the clamp 130 to be coupled to the housing 110 and allows the clamp 130 to pivot with respect to the housing 110. In one embodiment, the ring 131 is a key ring removably coupled to the clamp 130 and the loop 112. In one example, the clamp 120 and the clamp 130 may not extend laterally from the housing 110.

The device 100 may include the clamp 120 extending from a lateral surface for coupling the device 100 to an individual or an object. Similarly, the clamp 130 and the clamp 140 each extend from a lateral surface of the device 100 for coupling to a medical tube.

The clamp 120, in one embodiment, is a spring clamp that securely clamps onto an object. For example, the clamp 120 may be clamped onto clothing of a patient, an IV pole, an infusion pump, the patient's bed and the like.

The clamp 130 is configured to clamp onto a medical tube (e.g., an oxygen tube, an IV tube, and the like). In particular, the clamp 130 is configured to clamp on to a medical tube and firmly grip the medical tube without reducing the flow of fluid through the medical tubing. For example, the clamp 130 may slightly deform the medical tube by clamping around the outside diameter of the tube. However, the slight deformation of the outside diameter of the tube (and the inside diameter of the tube) does not reduce the flow or flow rate of the fluid flowing through the medical tube. Alternatively, the slight deformation of the outside diameter of the tube (and the inside diameter of the tube) negligibly reduces the flow or flow rate.

The clamp 140 is configured to clamp onto a medical tube (e.g., an oxygen tube, an IV tube, and the like). In particular, the clamp 140 is configured to clamp on to a medical tube and firmly grip the medical tube without reducing the flow of fluid through the medical tubing. For example, the clamp 140 may slightly deform the medical tube by clamping around the outside diameter of the tube. However, the slight deformation of the outside diameter of the tube (and the inside diameter of the tube) does not reduce the flow or flow rate of the fluid flowing through the medical tube. Alternatively, the slight deformation of the outside diameter of the tube (and the inside diameter of the tube) negligibly reduces the flow or flow rate. In one embodiment, the clamp 130 and the clamp 140 are the same type of clamp. In another embodiment, the clamp 130 and the clamp 140 are different types of clamps.

The clamp 130 may clamp on a medical tubing at a first location on the medical tubing, and the clamp 140 may clamp on the medical tubing at a second location on the medical tubing to create a loop in the medical tubing between the first location and the second location.

The FIG. 1C depicts a perspective view of the device 100, according to an embodiment. A perspective view may depict a three-dimensional image that portrays height, width, and depth of an object. FIG. 1C depicts a height, width, and depth of the device 100 that may not be depicted in at least the FIG. 1B. For example, the FIG. 1C, depicts a height, width, and depth of at least the clamp 120, the clamp 130, and the clamp 140. In one embodiment, clamp 130 includes grip feature 139 to increase a user's grip on clamp 130 when the user clamps clamp 130 onto a medical tube. In one embodiment, clamp 140 includes grip feature 149 to increase a user's grip on clamp 140 when the user clamps clamp 140 onto a medical tube.

Referring to the FIG. 1D, the clamp 140 may extend from the housing 110 via the cord 141, according to an embodiment. For example, in response to a force acting on the clamp 140, in the direction 143, the clamp 140 may extend from the housing 110. Upon a force not acting on the clamp 140 (while in the extended state), the clamp 140 is automatically retracted back into the housing 110 via a reel automatically winding the cord 141. In one embodiment, the clamp 140 may be extended from the housing 110 in range from approximately two feet to three feet. In another embodiment, the claim 140 may be extended from the housing 110 to approximately five feet.

The device 100 is a portable retractable device for management of medical tubing. T device 100 may be readily carried and transported by a hand of an individual for convenient and quick attachment to a medical tube and an object (e.g., individual, IV pole, infusion pump and the like). In one embodiment, the device 100 weighs in a range of 0.5 ounces to 5 ounces. In another embodiment, the device may weigh less than 0.5 ounces and/or more than 5 ounces.

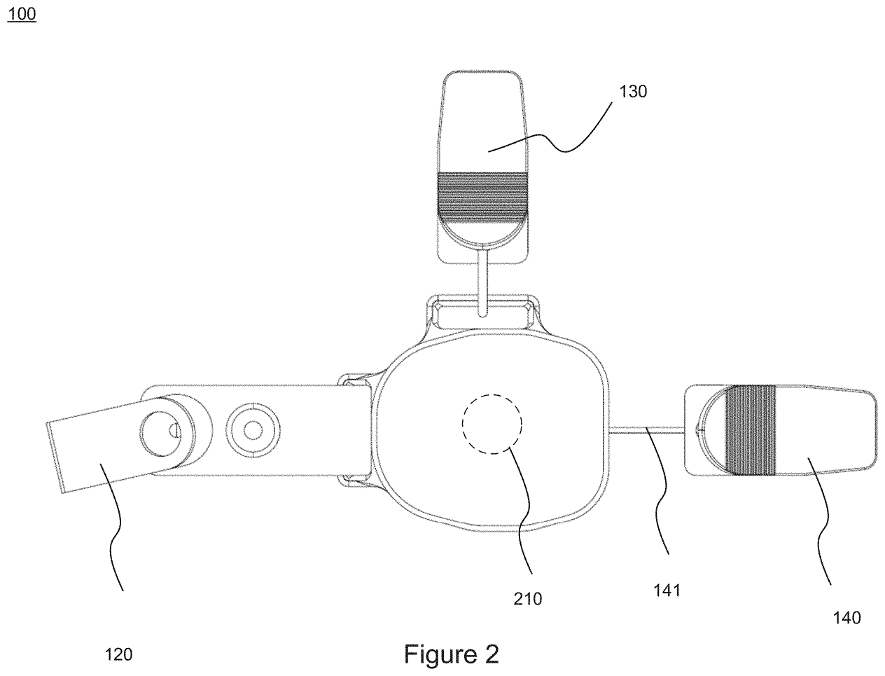

FIG. 2 depicts a top view of the device 100 that includes a depiction of the reel 210, according to an embodiment. As described herein, the retractable cord 141 is retracted within the housing 110 via a reel, such as the reel 210. For example, in a retracted state, cord 141 is wound around reel 210. In response to a pulling force on cord 141, cord 141 unwinds from around reel 210 and extends away from housing 110.

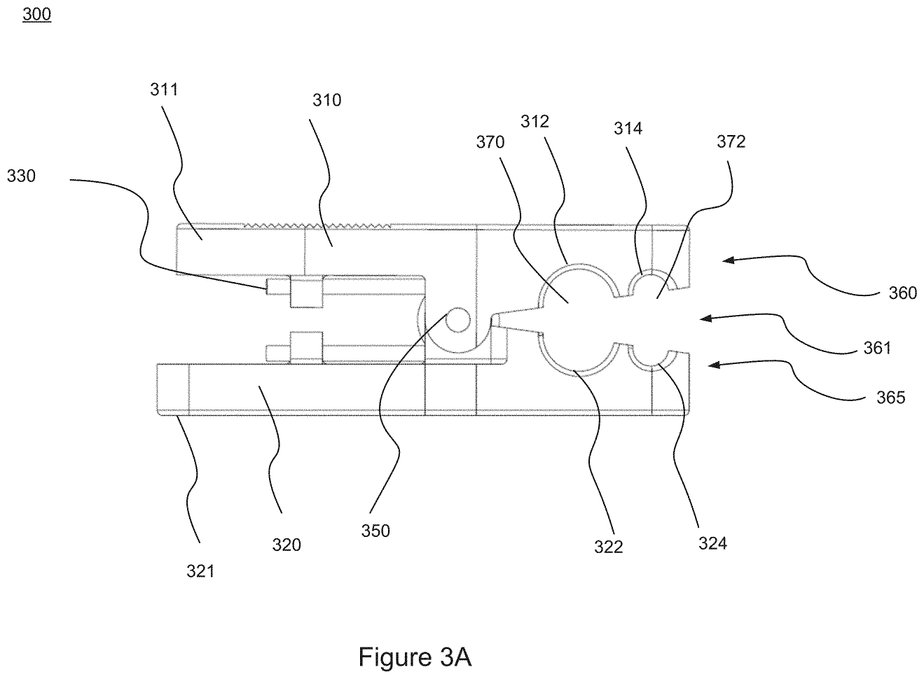

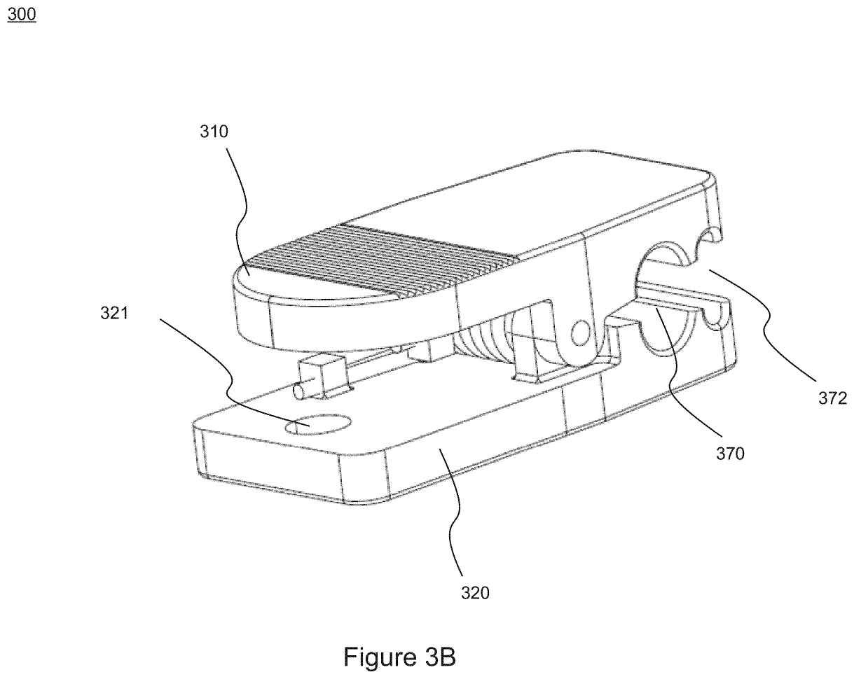

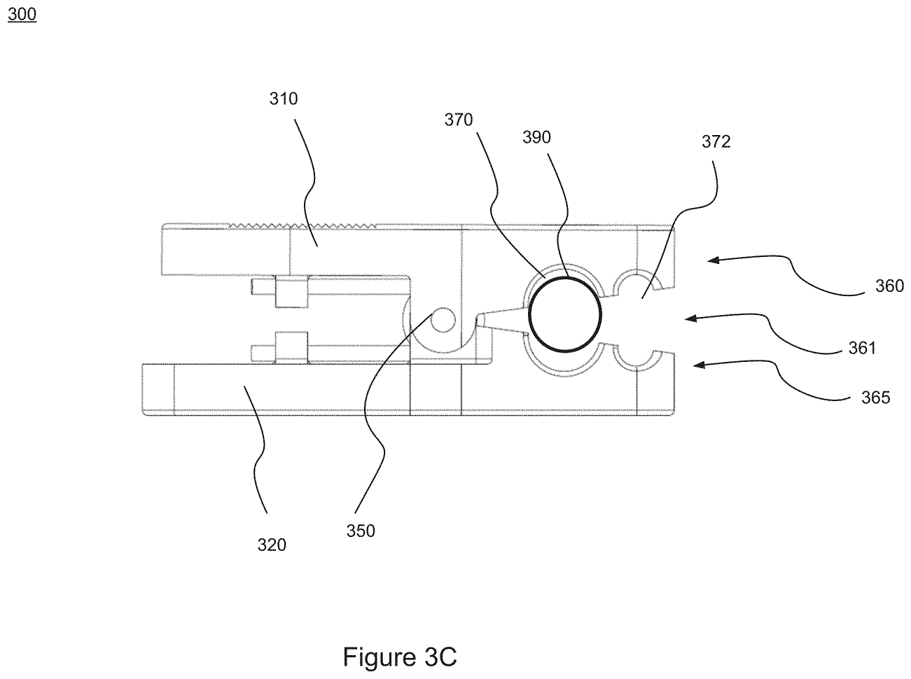

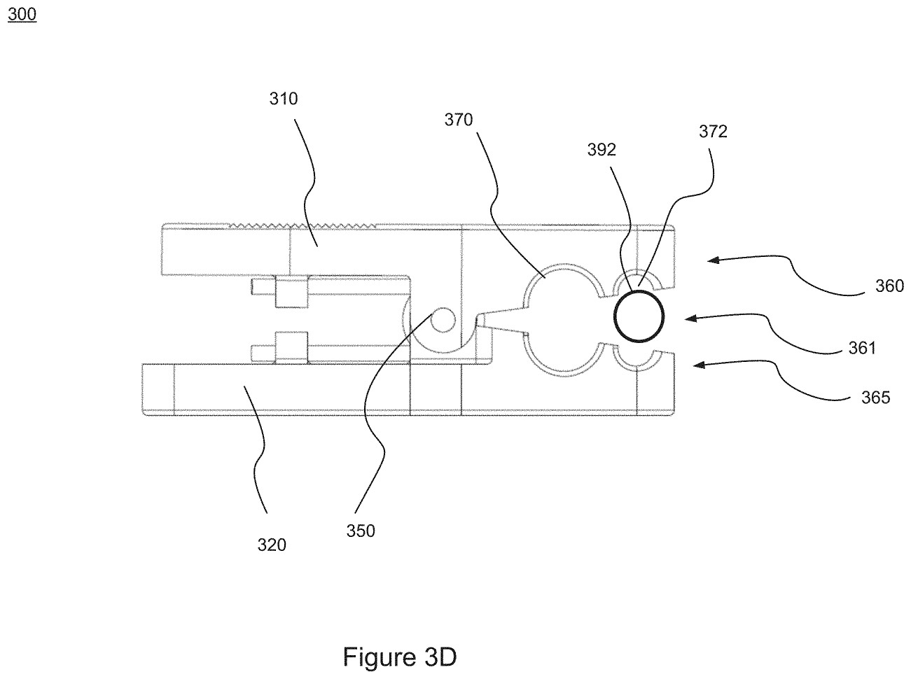

The FIG. 3A depicts the medical tube clamp 300 (also referred to herein as “clamp 300”), according to an embodiment. The FIG. 3A depicts a side view of the clamp 300. In one embodiment, the clamp 300 is a spring clamp. In another embodiment, the claim 300 is a pipe clamp. In various embodiments, the clamp 300 is an embodiment of the clamp 130 and/or the clamp 140.

The clamp 300 may include the first handle 310 (also referred to herein as “handle 310”) and the second handle 320 (also referred to herein as “handle 320”). The handle 310 and the handle 320 are coupled to each other about the hinge 350. As such, the handle 310 and the handle 320 pivot with respect to one another about the hinge 350.

The handle 310 includes the first jaw 360 (also referred to herein as “jaw 360”) and the handle 320 includes the second jaw 365 (also referred to herein as “jaw 365”). The jaw 360 and the jaw 365 are biased towards one another via the spring 330. In particular, the spring 330 is coupled to both the handle 310 and the handle 320 which biases the end 311 of the handle 310 away from the end 321 of the handle 320. When the spring 330 is in a relaxed state, the spring 330 biases the jaw 360 and the jaw 365 towards one another at the intersection 361. In one embodiment, the jaw 360 and jaw the 365 are biased together such that they physically contact each other at the intersection 361. In another embodiment, the jaw 360 and the jaw 365 are biased together such that they do not physically contact each other at the intersection 361.

The jaw 360 includes the cavity 312 and the jaw 365 includes the cavity 322. When the jaw 360 and the jaw 365 are biased together, the cavity 312 of the jaw 360 and the cavity 322 of the jaw 365 form the aperture 370. The aperture 370 is configured to clamp or grip a first medical tube having a first diameter.

Similarly, the jaw 360 includes the cavity 314 and the jaw 365 includes the cavity 324. When the jaw 360 and the jaw 365 are biased together, the cavity 314 of the jaw 360 and the cavity 324 of the jaw 365 form the aperture 372. The aperture 372 is configured to clamp or grip a second medical tube having a second diameter (smaller than the first diameter). More specifically, the aperture 372 is similar in size and shape to the outer diameter of the medical tubing. As such, the clamp is able to clamp around the medical tubing without deforming (or minimally) deforming the medical tube.

The FIG. 3B depicts a perspective view of the clamp 300, according to an embodiment. A perspective view may depict a three-dimensional image that portrays height, width, and depth of an object. As such, the FIG. 3B depicts a height, width, and depth of the clamp 300 that may not be depicted in at least the FIG. 3A. For example, FIG. 3B, depicts a height, width, and depth of clamp 300. In one embodiment, handle 320 includes hole 321. Hole 321 allows clamp 300 to be attached to device 100. For example, ring 321 is attached to hole 321 and attached to a loop (e.g., loop 112) of housing 110.

FIG. 3C depicts an embodiment of the clamp 300 clamping a first medical tube having a first diameter, according to an embodiment. The clamp 300 clamps the medical tube 390 within the aperture 370. In one embodiment, the aperture 370 has a diameter that is similar diameter (e.g., a slightly smaller diameter) to an outside diameter of the medical tube 390. As such, the medical tube 390 is clamped within the aperture 370 without overly deforming/pinching the medical tube. Therefore, the medical tube 390 is clamped within the aperture 370 without decreasing or inhibiting the flow of fluid through the medical tube 390. Alternatively, the medical tube 390 is minimally deformed and reduction in flow is negligible. In one embodiment, the tube 390 passes through an entire length of the aperture 370.

The FIG. 3D depicts the clamp 300 clamping a second medical tube having a second diameter (smaller than the first diameter). Referring to the FIG. 3D, the clamp 300 clamps the medical tube 392 within the aperture 372. In one embodiment, the aperture 372 has a diameter that is similar diameter (e.g., a slightly smaller diameter) to an outside diameter of the medical tube 392. As such, the medical tube 392 is clamped within the aperture 372 without overly deforming/pinching the medical tube. Therefore, the medical tube 392 is clamped within the aperture 372 without decreasing or inhibiting the flow of fluid through the medical tube 392. Alternatively, the medical tube 390 is minimally deformed and reduction in flow is negligible. In one embodiment, the tube 392 passes through an entire length of the aperture 372.

In one embodiment, the clamp 300 clamps a single tube at a time. For example, the clamp 300 clamps the tube 390 (and does not clamp the tube 392), or the clamp 300 clamps the tube 392 (and does not clamp the tube 390).

In another embodiment, the clamp 300 concurrently clamps separate tubes. For example, the clamp 300 concurrently clamps the tube 390 and the tube 392.

In a further embodiment, the clamp 300 includes the aperture 370 and another aperture) similar to the aperture 370 (e.g., another aperture of the same size—not shown. As such, the clamp 300 may concurrently clamp the tube 390 in the aperture 370 and another tube (similar to the tube 390) in the another aperture (similar to the aperture 370).

The medical tube 390 and/or the medical tube 392 may include various materials, such as but not limited to, polyvinyl chloride (PVC), silicon rubber, fluoropolymer, thermoplastic, thermoplastic elastomer, fluoroelastomer, and the like.

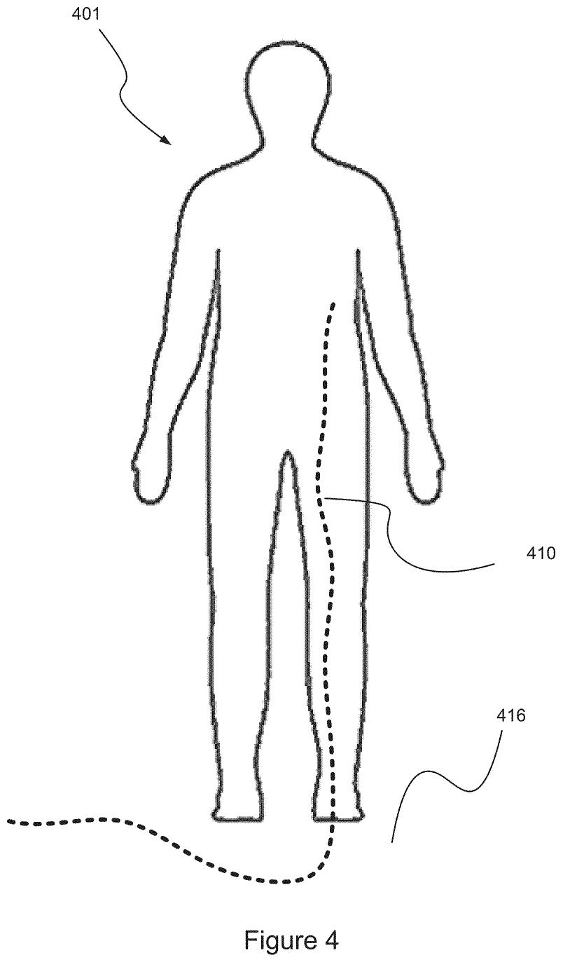

FIG. 4 depicts a medical tube (e.g., the medical tube 410) that may become entangled with the individual 401 (e.g., a patient), according to an embodiment. This may limit or prohibit fluid (e.g., medicine, oxygen, etc.) from passing through the medical tube 410 and administered to the individual 401. For example, a portion of the medical tube 410 lays on the ground surface 416. The portion of the medical tube on the ground surface 416 may trip the individual 401 or may become entangled with medical devices (e.g., IV pole).

FIG. 5A depicts an embodiment of the medical tubing management system 500 (also referred to the “system 500”), according to an embodiment. In particular, the FIG. 5A depicts the device 100 of the system 500 in a retracted state and the FIG. 5B depicts device 100 of system 500 in an extended state. In one embodiment, system 500 is similar to system 150 described herein.

The system 500 includes the medical tube 510 and the device 100. The medical tube 510, in one embodiment, is an IV tube coupled to the individual 501 at the location 502 (e.g., an IV port) of the individual. In particular, a first distal end of the tube 510 is coupled to the individual and a second distal end of the tube 510 is coupled to a medical device (e.g., an infusion pump, not shown). In another embodiment, the medical tube 510 is an oxygen tube coupled to the individual 501 proximate a mouth and/or nose of the individual (at a first distal end of the tube 510) and coupled to an oxygen pump or canister at an opposite second distal end of the tube (not shown).

The device 100 may be coupled to the individual 501 via the clamp 120. For example, the clamp 120 is coupled to clothing or a belt of the individual 501. The device 100 is coupled at a position between the location 502 and the ground surface 516.

The clamp 130 may be clamped to the tube 510 (at the location 518 of the tube 510). The clamp 130 can be clamped at any position of the tube 510 such that there is at least some slack of the tube 510 between the location 502 of the individual and the location 518. Accordingly, stress acting on the tube at the location 502 of the individual is limited.

The clamp 140 may be clamped to the tube 510 (at the location 519 of the tube 510). The loop 515 of the tube 510 is formed between the location 518 and the location 519 of the tube 510. The loop 515 is suspended above the ground surface 516. Accordingly, the loop 515 cannot be entangled at or near the ground surface 516. The clamp 140 may be clamped to the tube 510 at any location such that a loop is created (between the clamp 130 and the clamp 140) and suspended above the ground surface 516.

In one embodiment, at least a portion of the tube 510 may be on the ground surface 516 prior to formation of the loop 515. For example, prior to formation of the loop 515, a portion of the tube 510 lays along the ground surface 516 and causes a tripping or entanglement hazard at the ground surface 516.

FIG. 5B depicts a pulling force is acted on the tube 510 in the direction 570, according to an embodiment. For example, as an individual walks away from an infusion pump, this action may cause a pulling force on the tube 510 in the direction 570. Accordingly, the clamp 120 remains coupled to the individual 501 and the clamp 130 remains coupled to tube at the location 518.

Additionally, the clamp 140 extends away from the housing 110 of the device 100 via the cord 141. Accordingly, the pulling force is absorbed via the clamp 140 extending from the housing 110 (and the clamp 120 clamped to the individual and the clamp 130 clamped to the tube 510) and the pulling force does act on the tube 510 at the location 502 of the individual 501. For example, this prohibits the tube from pulling out of the individual at an IV port at the location 502. Moreover, the loop 515 remains suspended above the ground surface 516.

In response to the pulling force not acting on the tube 510 in the direction 570 (for example, the individual walks back towards the infusion pump), the clamp 140 is drawn towards the housing 110 of the device 100 via the cord 141 (see the FIG. 5A). Moreover, the loop 515 remains suspended above the ground surface 516.

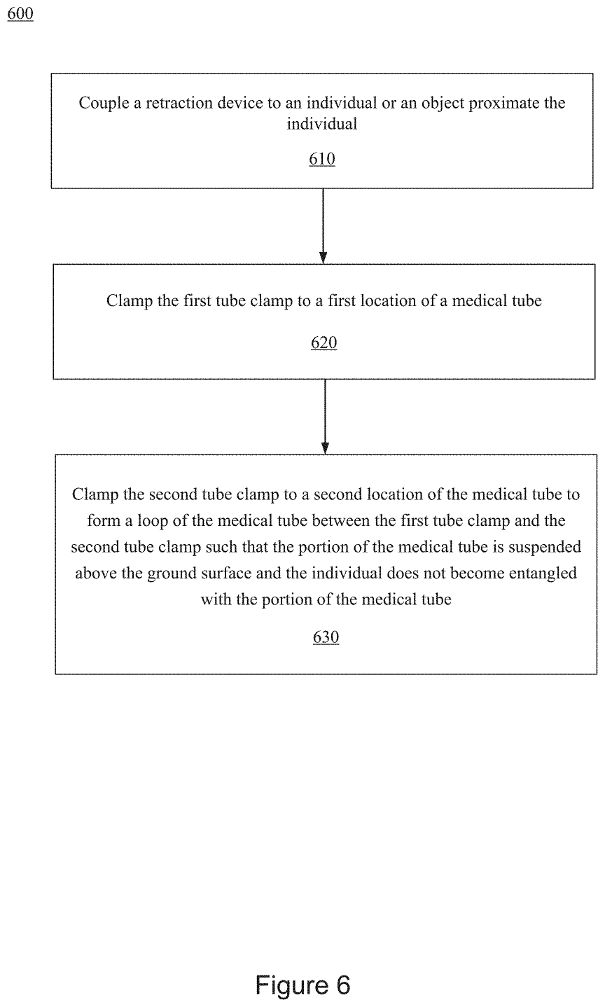

FIG. 6 depicts a method 600 for managing medical tubing, according to an embodiment. The method may include coupling a retraction device to an individual or an object proximate the individual (block 610). For example, referring to the FIG. 5A, the device 100 is coupled to the individual 501 via the clamp 120. The clamp 120 is releasably affixed to a garment worn by the individual 501. In another example, referring to the FIG. 1A, device 100 is coupled to pump 163 (that is coupled to IV pole 162).

The method 600 may include clamping a first tube to a first location of a medical tube (block 620). For example, referring to the FIGS. 3A and 5A, the aperture 370 of the clamp 300 (e.g., the clamp 130 of device the 100) clamps around the tube 510 (at the location 518 of the tube 510).

The method 600 may include clamping a second tube to a second location of the medical tube to form a loop of the medical tube between the first tube clamp and the second tube clamp (block 630). For example, referring to FIGS. 3A and 5A, the aperture 370 of the clamp 300 (e.g., the clamp 140 of the device 100) clamps around the tube 510 (at the location 519 of the tube 510). Accordingly, the loop 515 of the tube 510 is formed between the clamp 140 and the clamp 130 where the loop 515 is suspended above the ground surface 516. Moreover, because the loop 515 is suspended above the ground surface 516, the individual 501 does not become entangled with a portion of the tube 510 that forms loop the 515.

It is to be understood that the above description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The disclosure above encompasses multiple distinct embodiments with independent utility. While these embodiments have been disclosed in a particular form, the specific embodiments disclosed and illustrated above are not to be considered in a limiting sense as numerous variations are possible. The subject matter of the embodiments includes the novel and non-obvious combinations and sub-combinations of the various elements, features, functions and/or properties disclosed above and inherent to those skilled in the art pertaining to such embodiments. Where the disclosure or subsequently filed claims recite “a” element, “a first” element, or any such equivalent term, the disclosure or claims is to be understood to incorporate one or more such elements, neither requiring nor excluding two or more such elements.

Applicant(s) reserves the right to submit claims directed to combinations and sub-combinations of the disclosed embodiments that are believed to be novel and non-obvious. Embodiments embodied in other combinations and sub-combinations of features, functions, elements and/or properties may be claimed through amendment of those claims or presentation of new claims in the present application or in a related application. Such amended or new claims, whether they are directed to the same embodiment or a different embodiment and whether they are different, broader, narrower or equal in scope to the original claims, are to be considered within the subject matter of the embodiments described herein.

1. A portable retracting device for medical tubing management, comprising:
a housing comprising:
a first side;
a second side opposite the first side;
a third side extending between the first side and the second side; and
a fourth side opposite the third side, wherein the first side, the second side, the third side and the fourth side define a periphery of the housing; and
a retractable cord disposed within the housing, wherein the retractable cord is configured to extend from the first side of housing;
a first spring clamp affixed to the second side of the housing configured to attach to clothing of an individual, wherein a first medical tube is attached to:
the individual at a first end of the first medical tube; and
a pump at a second end of the first medical tube;
a second spring clamp affixed to the third side of the housing, wherein the second spring clamp comprises:
a first jaw; and
a second jaw, wherein:
the first jaw and the second jaw physically engage with one another at a first intersection; and
the first intersection comprises a first aperture configured to clamp to the first medical tube having a first diameter at a first location of the first medical tube; and
a third spring clamp coupled to an end of the retractable cord and disposed proximate the first side of the housing, wherein the third spring clamp comprises:
a third jaw; and
a fourth jaw, wherein:
the third jaw and the fourth jaw physically engage with one another at a second intersection;
the second intersection comprises a second aperture configured to clamp the first medical tube at a second location of the first medical tube to form a first loop in the first medical tube from the first location of the first medical tube to the second location of the first medical tube;
a portion of the first medical tube that forms the first loop does not contact a ground surface such that the individual does not become entangled with the portion of the first medical tube at the ground surface; and
in response to the individual generating a pulling force on the first medical tube in a direction from the first end of the first medical tube to the second end of the first medical tube:
the first spring clamp is configured to remain affixed to the clothing of the individual;
the second spring clamp is configured to remain clamped to the first medical tube at the first location;
the third spring clamp is configured to:
remain clamped to the first medical tube at the second location;
extend away from the housing via the retractable cord;
the portion of the first medical tube is configured to continue to not contact a ground surface;
the first end of the first medical tube is configured to remain attached to the individual; and
the second end of the first medical tube is configured to remain attached to the pump.

2. The portable retracting device of claim 1, wherein, in response to the individual moving towards the pump, when the retractable cord is extended from the housing, and the pulling force is not acting on the first medical tube:
the first spring clamp is further configured to remain affixed to the clothing of the individual;
the second spring clamp is further configured to remain clamped to the first medical tube at the first location,
the third spring clamp is further configured to:
remain clamped to the first medical tube at the second location; and
move towards the housing via the retractable cord;
the portion of the first medical tube is further configured to continue to not contact a ground surface;
the first end of the first medical tube is further configured to remain attached to the individual; and
the second end of the first medical tube is further configured to remain attached to the pump.

3. The portable retracting device of claim 1, wherein:
the first aperture comprises a second diameter smaller than the first diameter of the first medical tube;
the second aperture comprises the second diameter; and
the first medical tube is configured to:
pass through a first length of the first aperture at the first location; and
pass through a second length of the second aperture at the second location.

4. The portable retracting device of claim 1, wherein the first spring clamp further comprises:
a first handle comprising the first jaw;
a second handle opposite the first handle, wherein:
the second handle comprises the second jaw; and
the first handle and the second handle are hingably coupled to one another at a hinge; and
a spring disposed proximate the hinge and coupled to the first handle and the second handle.

5. The portable retracting device of claim 1, wherein:
the first jaw comprises a first cavity; and
the second jaw comprises a second cavity, wherein the first cavity and the second cavity form the first aperture.

6. The portable retracting device of claim 1, wherein:
the first intersection further comprises a third aperture configured to clamp to a second medical tube at a third location of the second medical tube;
the second medical tube having a third diameter; and
the second intersection further comprises a fourth aperture configured to clamp to the second medical tube at a fourth location of the second medical tube to form a second loop in the second medical tube from the third location of the second medical tube to the fourth location of the second medical tube.

7. The portable retracting device of claim 1, wherein the first medical tube is an intravenous (IV) tube or an oxygen tube.

8. A medical tubing management system, comprising:
a medical tube comprising:
a first end coupled to an intravenous (IV) port of an individual; and
a second end coupled to an intravenous (IV) bag comprising fluid, wherein the individual is fluidically coupled to the IV bag to receive the fluid in the IV bag an IV device coupled to the IV bag; and
a retraction device comprising:
a coupling device configured to couple the retraction device to the IV device;
a first tube clamp configured to clamp the medical tube at a first location of the medical tube;
a retractable cord disposed within the retraction device; and
a second tube clamp coupled to the retractable cord, the second tube clamp is configured to clamp the medical tube at a second location of the medical tube to form a loop in the medical tube between the first tube clamp and the second tube clamp, wherein a portion of the medical tube that forms the loop does not contact a ground surface such that the individual does not become entangled with the loop at the ground surface; and
in response to the individual generating a pulling force on the medical tube in a direction from the first end of the medical tube to the second end of the medical tube:
the coupling device is configured to remain coupled to the IV device;
the first tube clamp further configured to remain grasped to the medical tube at the first location; and
the second tube clamp further configured to:
remain grasped to the medical tube at the second location; and
extend away from the retraction device via the retractable cord;
the loop continues to not contact the ground surface; and
the individual remains fluidically coupled to the IV bag to receive the fluid in the IV bag.

9. The medical tubing management system of claim 8, wherein, in response to the individual moving towards, when the retractable cord is extended from the retraction device, and the pulling force is not acting on the medical tube:
the coupling device is further configured to remain coupled to the IV device;
the first tube clamp is further configured to remain clamped to the medical tube at the first location; and
the second tube clamp further configured to:
remain clamped to the medical tube at the second location; and
retract towards the retraction device via the retractable cord;
the loop continues to not contact the ground surface; and
the individual remains fluidically coupled to the IV bag to receive the fluid in the IV bag.

10. The medical tubing management system of claim 8, wherein:
the retractable cord is configured to extend a first length from the retraction device; and
the loop of the tube having a second length that is greater than the first length.

11. The tube management system of claim 11, wherein the first length is a range between two feet and three feet.

12. The medical tube management system of claim 8, wherein the retraction device is coupled to the individual between the IV port and the ground surface

13. The medical tube management system of claim 8, wherein the coupling device is coupled to an IV pole or an infusion pump fluidically coupled to the IV bag.

14. The medical tube management system of claim 8, wherein:
the coupling device is coupled to the retraction device at a third location of the retraction device; and
the second tube clamp is disposed at a fourth location of the retraction device, wherein the third location is opposite the fourth location.

15. The medical tube management system of claim 14, wherein first tube clamp is coupled to the retraction device at a fifth location of the retraction device different than the third location and the fourth location.

16. The medical tube management system of claim 8, wherein:
the first tube clamp is disposed at a first wall of the retraction device; and
the second tube clamp is disposed at a second wall of the retraction device, wherein an angle between the first wall and the second wall is 90°.

17. A method, comprising:
coupling a retraction device to an individual or an object proximate the individual, wherein the retraction device comprises:
a first tube clamp; and
a second tube clamp retractably coupled to a retractable cord of the retraction device, wherein:
a medical tube is coupled to the individual at a first end the medical tube;
the medical tube is coupled to a medical pump at a second end of the medical tube; and
a portion of the medical tube is disposed on a ground surface that creates a risk of entanglement of the individual with the portion of the medical tube at the ground surface;
clamping the first tube clamp to a first location of a medical tube; and
clamping the second tube clamp to a second location of the medical tube to form a loop of the medical tube between the first tube clamp and the second tube clamp such that:
the portion of the medical tube is suspended above the ground surface; and
the individual does not become entangled with the portion of the medical tube.

18. The method of claim 17, further comprising, in response a pulling force acting on the medical tube in a direction from the first end to the second end:
extending the first tube clamp away from the retraction device via the retractable cord; and
maintaining suspension of the portion of the medical tube above the ground surface.

19. The method of claim 18, wherein, in response to the pulling force not acting on the medical tube in the direction from the first end to the second end:
retracting the first tube clamp towards the retraction device; and
maintaining the suspension of the portion of the medical tube above the ground surface.

20. The method of claim 18, wherein the retraction device is clamped to the object proximate the individual at a location between an intravenous (IV) bag coupled to the medical tube and the ground surface.
